(19) United States
(12) Patent Application Publication
(10) Pub. No.: US 2005/0240068 A1
(43) Pub. Date: Oct. 27, 2005

(54) Dephlegmatic phased method of organic waste utilization and dephlegmatic pyrolysis apparatus

(76) Inventor: Victor Petrovich Babiy, Nikolaev (UA)

Correspondence Address: ANTHONY R. BARKUME, 20 GATEWAY LANE, MANORVILLE, NY, 11949, US

(21) Appl. No.: 11/104,064

(22) Filed: Apr. 11, 2005

(30) Foreign Application Priority Data
Apr. 22, 2004 (UA) 20040403022

(51) Int. Cl. C07C 1/00; C10G 1/00

(52) U.S. Cl. 585/241

(57) ABSTRACT

This invention relates to a method for thermal utilization of solid organic household and industrial waste, and may be applied in municipal economy, chemical, petrochemical and other branches of industry for the regeneration of hydrocarbons into fuel oil and fuel gas. The dephlegmatic phased method of organic waste utilization, comprising multistage pyrolysis of waste in a reactor without air access, at temperature not more than 1000° with obtaining of solid carbon residue and multi-component gas-vapor mixture, cooling of gas-vapor mixture in multiloop circulatory cooling system, extraction of a heavy fraction for repyrolysis and obtaining of a liquid fraction having indicated molecular mass and gas on a finite circuit. Organic waste are exposed stepwise to the pyrolysis with reloading of non-decomposed solid residue from a retort of the first stage reactor having temperature 150-180° C., sequentially to retorts for the pyrolysis at higher temperatures, and gas-vapor mixture from the retorts is directed to not less than one dephlegmator, there it is purified from tar composition, and tar composition being returned to a retort for the re-pyrolysis, gas-vapor mixture from the dephlegmator being conveyed to a fraction distributor for cooling, and combustion gases from a casing of the last retort being conveyed to casings of the preceding retorts, and then jointly with combustion gases generated by heaters under the casings of all retorts are delivered outside. The attained object is the provision of an apparatus for the pyrolysis of improved effectiveness, obviating overheating and reheating of the finished product, and providing for obtaining of qualitative oil and gas fuel and pyrographite.

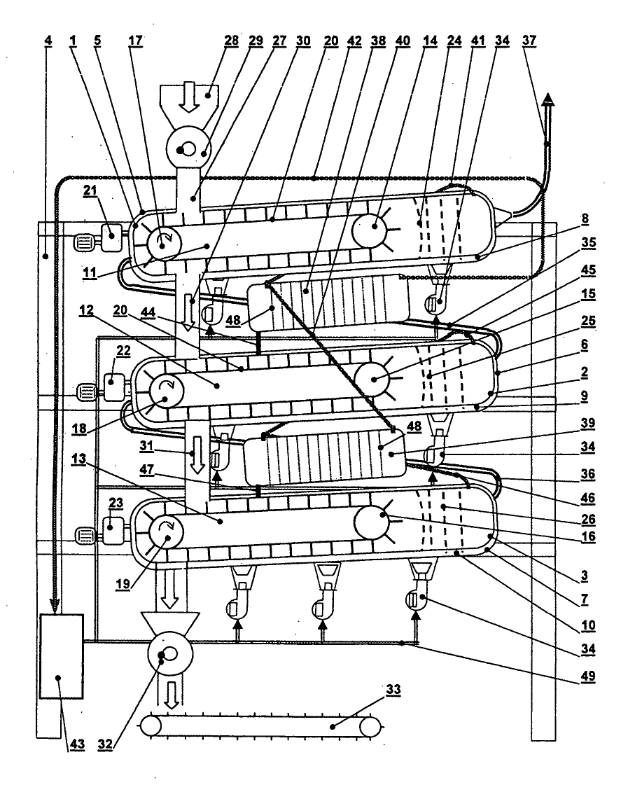

This application is based on Ukrainian Patent Application Serial No. 2240403022 filed into the Ukrainian Patent Office on Apr. 22, 2004.

This invention relates to a method for thermal utilization of solid organic household and industrial waste, and may be applied in municipal economy, chemical, petrochemical and other branches of industry for regeneration of hydrocarbons into fuel oil and fuel gas.

It has previously been proposed obtaining of low olefins by the pyrolysis of hydrocarbons, described in U.S. Pat. No. 45,503 of Ukraine, MPK6 C10G9/00, 9/36, F28D7/10, and published on Apr. 15, 2002 in Bull. No. 4/2002, comprising warming and vaporizing of initial raw material, its mixing with steam-diluent, heating of mixture in a spade reactor to pyrolysis temperature, cooling of pyrolysis gases and their further separation, at that heating of mixture to pyrolysis temperature is being affected by mixing with hot pyrolysis gases circulating in a working cavity of the spade reactor over the time far less in comparison with the pyrolysis reaction time. Warming of raw material and steam-diluent is being effected in two stages, where at the second stage warming is being effected in a heat exchanger at the cost of utilization of heat containing in pyrolysis gases flowing out of the spade reactor.

Features coinciding with the essential features of the method claimed: warming and vaporizing of initial raw material, heating of mixture to pyrolysis temperature, cooling of pyrolysis gases and their further separation.

Reasons, impeding obtaining of a necessary technical result:

There are particular disadvantages of the method presented: low economy, because of necessity to supply steam-diluent to raw material up to 100% of water to hydrocarbons weight, for which big heat consumptions are required, and upon completion of the process obtained liquid and gas are to be drained (dehydrated). Increased noisiness of a rotary drive gas turbine engine. High value of an installation production, which have expensive and hard to maintain components.

It has previously been proposed an organic waste utilization method, described in U.S. Pat. No. 52,840 of Ukraine, MPK7 F23G5/027, C10G1/00, and published on Jan. 15, 2003 in bull. No. 1/2003, comprising pyrolysis of waste in a reactor without air access at temperature 400-980° with obtaining of solid carbon residue and multi-component gas-vapor mixture, its further cooling in multiloop circulatory cooling system having design number of circuits and temperatures of coolants, extraction of a heavy liquid fraction and obtaining of a liquid fraction having indicated molecular mass on a finite circuit, during process execution control of values of molecular mass M of a liquid fraction is being carried out, which was obtained on a cooling system finite circuit, when M>150 the temperature of coolants for each circuit starting with the first, is reduced from the calculated by 50° C. maximum, and at M<150 is increased by 50° C. starting with the finite circuit, and if M<150 value did not change, then circuits number is reduced by alignment of adjacent circuits temperatures starting with first circuit temperature.

Features coinciding with the essential features of the method claimed: multistage pyrolysis of waste in a reactor without air access at temperature not more than 1000° with obtaining of solid carbon residue and multi-component gas-vapor mixture, cooling of gas-vapor mixture in multiloop circulatory system, extraction of a heavy liquid fraction for the re-pyrolysis and obtaining of a liquid fraction having indicated molecular mass and gas on a finite circuit.

Low economy is a disadvantage of the method presented, because of necessity of forced cooling of each reactor circuit, no heat recuperation, which considering working temperature up to 1000° C. leads to heat excessive consumption.

An object of the invention is to enhance effectiveness of the organic waste utilization method, to obviate overheating and re-heating of the finished product, to provide for obtaining of qualitative oil and gas fuel and pyrographite.

The dephlegmatic phased method of organic waste utilization according to the invention includes multistage pyrolysis of waste in a reactor without access for air at temperature not more than 1000° with obtaining of solid carbon residue and multi-component gas-vapor mixture, cooling of gas-vapor mixture in multiloop circulatory system, extraction of a heavy fraction for the re-pyrolysis and obtaining of a liquid fraction having indicated molecular mass and gas on a finite circuit.

Organic waste are exposed stepwise to the pyrolysis with reloading of non-decomposed solid residue and tar composition, which condensed on a dephlegmatic lattice for primary purification of a retort of the first stage, having temperature 180-250° C., sequentially to the retort for pyrolysis of the second stage at temperature 300-400° C., wherefrom non-decomposed solid residue and tar composition being conveyed for pyrolysis of the third stage at temperature 500-900° C., and gas-vapor mixture from the retort of the first stage is being conveyed to the first dephlegmator, purified from tar composition, and tar composition being conveyed to the retort of the second stage. Gas-vapor mixture from the second and the third stages retorts is being purified on dephlegmatic lattices for retorts primary purification and directed to the second dephlegmator, where it is additionally purified from tar composition, tar composition is being conveyed to the retort of the third stage, and purified gas-vapor mixture is being directed to the first dephlegmator, purified also from tar composition and jointly with gas-vapor mixture from the first stage retort being conveyed to a fraction distributor, wherein it is being cooled and segregated to liquid with molecular mass of 150-200 units and gas, and combustion gases from a cavity between a casing and the third stage retort are being conveyed under a second stage retort casing, return heat, then jointly with combustion gases generated by heaters under the second stage retort casing are being conveyed under the first stage retort casing, return heat and jointly with combustion gases generated by heaters of the first stage retort are being delivered outside, or to incineration and purification devices.

From the fraction distributor gas is conveyed to a reservoir for collection and used for retort heating burners, liquid is transferred to reservoirs and solid residue from the last retort is conveyed for cooling and to a packing shop through a discharging device. Combustion gases are directed for heat recovery, for example, in a destructor.

As opposed to a prototype, organic waste are being exposed sequentially to the pyrolysis with reloading of non-decomposed solid residue from the retort of the first stage reactor having temperature 180-250° C., to the second stage retort for the pyrolysis at temperature 390-400° C., wherefrom non-decomposed solid residue is being conveyed to the third stage retort for the pyrolysis at temperature 500-900° C., and gas-vapor mixture from the first stage retort is being conveyed to the first (low-temperature) dephlegmator, is purified from tar composition, and tar composition being conveyed to the retort of the second stage, gas-vapor mixture from the retorts of the second and the third stages arrives to the second dephlegmator, is purified from tar composition, tar composition is being conveyed to the third stage retort, and purified gas-vapor mixture enters the first dephlegmator, is purified also from tar composition and jointly with gas-vapor mixture from the first stage retort being conveyed to the fraction distributor, in which it is being cooled and segregated to liquid with molecular mass of 150 units and gas, and combustion gases from the third retort casing enter the second retort casing, return heat, then jointly with combustion gases generated by heaters under the second stage retort casing proceed under the first stage retort casing, return heat and jointly with combustion gases generated by heaters of the first retort are delivered outside.

From the fraction distributor gas is conveyed to the reservoir for collection and used for retort heating burners, liquid is transferred to reservoirs and solid residue from the last retort is conveyed for cooling and to a packing shop through a discharging device. Combustion gases are being directed for heat recovery, for example, in destructor, and to purification outside the aggregate.

Features essential in all cases are that organic waste are exposed stepwise to the pyrolysis with reloading of non-decomposed solid residue from the retort of the first stage reactor having temperature 180-250° C., sequentially to the retorts for the pyrolysis at higher temperatures, and gas-vapor mixture from the retorts is directed to not less than one dephlegmator, there it is purified from tar composition, and tar composition being returned to a retort for the re-pyrolysis, gas-vapor mixture from the dephlegmator being conveyed to the fraction distributor for cooling, and combustion gases from the last retort casing being conveyed to casings of the preceding retorts, and then jointly with combustion gases generated by heaters under the casings of all retorts are delivered outside.

Features essential in separate cases are that non-decomposed solid residue in the retort of the first stage reactor having temperature 180-250° C., being conveyed to the second stage retort for the pyrolysis at temperature 390-400° C., wherefrom non-decomposed solid residue is being conveyed to the third stage retort for the pyrolysis at temperature 500-900° C., and gas-vapor mixture from the first retort is being conveyed to the first dephlegmator, is purified from tar composition, and tar composition being conveyed to the retort of the second stage, and gas-vapor mixture from the second and the third stages retorts arrives to the second dephlegmator, is purified from tar composition, tar composition is being conveyed to the retort of the third stage, and purified gas-vapor mixture from the second dephlegmator being introduced to the first dephlegmator, is purified from tar composition, and tar composition being conveyed to the retort to the second stage.

In the retort of the second stage gas-vapor mixture is also purified on the dephlegmatic lattices of the retort, tar composition is being conveyed to the third stage retort, gas-vapor mixture is being conveyed onto the dephlegmatic lattices of the first stage retort, tar composition obtained is being returned to the second stage retort.

From the fraction distributor gas is conveyed to the reservoir for collection and used for retort heating burners, liquid is transferred to reservoirs and solid residue from the last retort is conveyed for cooling and to a packing shop through a discharging device. Combustion gases are being directed for heat recovery and to purification outside the aggregate.

Thus, population of processes provides for:
improvement of effectiveness of organic waste utilization due to application of heat of effluent combustion gases from the retorts heated more for heating of retorts of the pyrolysis stages which are carried out at lower temperatures;
overheating is excluded, and effectiveness is improved due to stepwise heating, starting with temperatures about 250° C., at which the pyrolysis of the lightest fractions, being directed at once for purification and segregation, is carried out;
at the output a qualitative product is obtained, as optimal temperature is ensured for the pyrolysis of basic spectrum of organic waste fractions and the re-pyrolysis of heavy fractions is carried out loss-free and at temperature approximate to optimal pyrolysis temperature for each fraction.

It is known a pyrolysis plant for organic waste utilization, described in declaration U.S. Pat. No. 36,635A of Ukraine, MPK6 F23G5/027A, published on Apr. 16, 2001 in Bull. No. 3/2001, comprising a hermetic reactor having external heating, axle with rotor, agitators, feeding and discharging devices, a conduit for gas-vapor mixture outlet, a rotor is made of separate sections in the form of a squirrel cage, which longitudinal ribs are equipped with resilient members staggered along the perimeter of a section and installed with possibility of elastic contact with inner reactor surface, and agitators are made in the form of a narrowing spiral tape and is located on the inner surface of each section. The sections are located along one axle and shifted one related to the other at an angle 90° from the beginning of the agitator spiral tape, where the agitators are located in the upper and lower sections so, that the spiral tape beginning is made as the narrow part and with right approach, and the last one as the wide part of the tape having left approach, and from the outer side of the lower section end there is a scraper installed around the axle, twisted with right approach in the form of Archimedes spiral.

Features coinciding with the essential features of the analogue are that the device comprises a hermetic reactor having external heating, agitators, feeding and discharging devices, a conduit for gas-vapor mixture outlet.

Disadvantages of the arrangement described are absence of heat recovery for needs of the plant itself, and one-stage processing, which leads to overheating of light constituents and impossibility to obtain homogeneous fractions of the necessary molecular weight.

The closest in the technical nature is the pyrolysis apparatus for waste processing, described in the declaration Patent of Ukraine for the utility model No.1484, MPK6 F23G5/027, F23G7/00, F23G7/12, published on Nov. 15, 2002 in Bull. No. 11/2002, and comprising the pyrolysis reactor located in the combustion chamber body, a feeding device equipped with sequentially located loading tray and a waste feeding auger, a system of burners which is placed in the combustion chamber, gas main line having feeding facilities of the system of burners from an external gas source, extraction of combustion gases from the combustion chamber body and of pyrolysis gases from the pyrolysis reactor, a discharging device having an auger for the product outlet. The pyrolysis reactor is installed along the combustion chamber and comprises a rigidly embedded in its block walls reactor body which is constructed in the form of a longitudinal pipe reinforcing rib-supported, and reinforcing ribs are placed in the combustion chamber body transversely to the reactor body, an auger shaft placed along the reactor body and installed in the combustion chamber body on bushings and made with the possibility of waste moving from the filling zone to the discharging zone for the time of a process cycle necessary for their complete sublimation, the discharging device comprises a transfer chamber having volume not less, than volume of obtained pyrographite at the end of the process cycle, which chamber is located in front of the auger for waste outlet, the transfer chamber diaphragm is installed transversely to direction of the processed raw materials moving from an unloading bunker and is made with the possibility of its opening on completion of full treatment cycle, an outlet mean for pyrolysis gases has a valve may be opened in case of exceeding pressure in the reaction zone above meanings necessary to exercise pyrolysis reaction, waste feeding augers, an outlet of the product and the pyrolysis reactor have equal geometric sizes, moreover, the apparatus is equipped with an operation auto-control unit, comprising an auger moving control unit, a control unit of air-mixture supply to the system of burners, a gas-distribution system unit, pressure and temperature sensors, auger moving speed sensors, where parts of the apparatus which may be affected by high temperatures are made of heat-proof and resistant to corrosive environment material, bodies of the combustion chamber and the feeding and discharging devices have heat insulation, and bushings are made of heat-insulating material.

An outlet of the gases withdrawal device from the pyrolysis reactor is connected to the feed system of burners.

General features are that the apparatus comprises a pyrolysis reactor with external heating, a feeding device, a system of burners located in the combustion chamber, gas main line having feeding facilities of the system of burners from an external gas source, extraction of combustion gases from the combustion chamber body and of the pyrolysis gases from the pyrolysis reactor, a discharging device. A reactor body is constructed in the form of a longitudinal pipe and comprises a waste moving device from the filling zone to the discharging zone for the time of the process cycle. The apparatus is equipped with an operation auto-control unit, comprising a raw material supply unit, a control unit of air-mixture supply to the system of burners, a gas-distribution system unit, temperature sensors, where elements parts of the apparatus amenable to high temperatures action are made of heat-proof and resistant to corrosive environment material, bodies of the combustion chamber and the feeding and discharging devices have heat insulation, and bushings are made of heat-insulating material.

The essential general features are that the apparatus comprises a pyrolysis reactor with external heating, a feeding device, a system of burners, gas main line having feeding facilities of the system of burners, system for withdrawal of combustion gases from the heating system and of pyrolysis gases from the pyrolysis reactor, a discharging device. The reactor comprises a device for waste moving from the filling zone to the discharging zone for the time of a process cycle, parts of the apparatus amenable to high temperatures action are made of heat-proof and resistant to corrosive environment material.

Disadvantages of the arrangement described are absence of heat recovery for needs of the plant itself, and one-stage processing, which leads to overheating of light constituents, gumming-up and impossibility to obtain homogeneous fractions of the necessary molecular weight.

An object of this invention is to provide an apparatus for pyrolysis having improved economy, elimination of overheating and reheating of the finished product, providing for obtaining of qualitative oil and gas fuel and pyrographite.

A dephlegmatic pyrolysis apparatus consists of three cylindrical retorts which are assembled on a frame upon 20°-40° tilt of cylinder generatrix relative to horizon, inside external casings, also cylindrical. There are conveyers having movable and drive pulleys inside each retort. Lobes of stainless heat-resistant steel are fastened to a conveyer band, the lobes in their height reach the walls of the retort body, having a gap not more than 0,5 mm. The conveyer is being set in motion with the help of a drive pulley, which shaft from the outer side of the retort and the casing is connected to a reducer, which is being set in motion by a synchronous or an induction motor changing shaft speed. The movable and the drive pulleys are installed inside the retort on the bearings with bushings. Inside the retorts there are also assembled dephlegmators (tar extractors) of primary purification located behind the movable pulley and consist of 4-5 pierced steel membranes being installed across the retort cylinder from the elevated side.

There is an opening made from the conveyor drive side, over which a feeding device is installed for feeding of crushed waste to the first stage retort. In the low part of each retort there is an aperture for reloading of solid residue to the next processing stage into the following retort, out of the second retort waste move either outside, or to the following retort for the third stage of processing. In the last retort there is a locking discharging device for unloading of pyrographite, and a cooling chamber with a conveyer being placed in a conveyor gallery, and supplying pyrographite to a sorting and packing shop.

The outer retort casing, in which an inner retort is installed, is 100 mm bigger. The outer side of each retort is finned along the whole length of the cylinder generatrix for a better heat-transmission. On the outer casing of each retort there are three gas burners installed and block units having slide automatic power control by set temperature in the retort, and three burners for fuel oil with three-stage power control. Cavities between the retorts and outer housings are connected between them sequentially with pipes, and a smoke tube is diverted from the first stage retort casing.

Between the retorts there are two dephlegmators installed which are attached to the cavities of the retorts with pipes and connected between them:
the first dephlegmator is connected to the second one and to the first stage retort with pipes for gas-vapor mixture and from it a pipe goes for supply of gas-vapor mixture to a cooler, and from the lowest place a pipe is diverted to the second stage retort cavity for tar composition;
the second dephlegmator is connected to the first dephlegmator, the second and the third stage retorts with pipes for gas-vapor mixture, and from the lowest place a pipe is diverted to the third stage retort cavity for tar composition.

The dephlegmators are cylinder reservoirs closed from the ends, assembled at an angle of 20°-40° to horizon, wherein there are metal membranes with a lot of apertures installed.

The essential distinctive features in all cases are that the pyrolysis apparatus comprises not less than two cylindrical retorts having close ends which are assembled on a frame inside external casings forming a furnace cavity, upon 20°-40° tilt of cylinder generatrix relative to horizon, and not less than one dephlegmator which is a cylinder closed from ends, assembled at an angle of 20°-40° relative to horizon, wherein numerous pierced metal membranes are installed across the cylinder, inside each retort there are conveyers with movable and drive pulleys, lobes are fastened to a conveyer band raking up raw material from the cylindrical surface of the retort body, an orifice for reloading of solid residue to the next stage of processing into the following retort is in the lowest part of each retort, the dephlegmator is fastened to retorts cavities with pipes.

The essential distinctive features in all cases are that inside the retorts behind the movable pulley across the retort cylinder from the elevated side of the retort there are dephlegmatic lattices for primary purification installed, consisting of 4-5 metal membranes with a lot of through apertures. On a frame there are three retorts installed and two dephlegmators which are fastened with pipes to retorts cavities and connected between them, where the first dephlegmator is connected to the second one and to the first stage retort with pipes for gas-vapor mixture and a pipe goes from it conveying gas-vapor mixture to a cooler, from the lowest place a pipe is diverted to the second stage retort cavity, and the second dephlegmator is connected to the first dephlegmator, the second and the third stage retorts, with pipes for gas-vapor mixture, and from the lowest place a pipe is diverted to the third stage retort cavity. On the outer casing of each retort there are furnaces assembled, on which gas burners are placed, block units having slide automatic power control according to the set temperature in the retort, and burners for fuel oil with three-stage power control, cavities between the retorts and outer casings are connected between them sequentially with pipes, and a smoke tube is diverted from the first stage retort casing. The outer side of the retort is finned along the whole area of the retort cylinder generatrix.

The dephlegmatic pyrolysis apparatus is disclosed in the figure in a diagram form.

The dephlegmatic pyrolysis apparatus consists of three cylindrical retorts: a retort of the first stage 1, a retort of the second stage 2, and a retort of the third stage 3, which are assembled on the joint frame 4 inside the outer cylindrical casings 5, 6 and 7, forming heating cavities 8, 9 and 10. The retorts are assembled upon 30° tilt of cylinder generatrix relative to horizon. Inside each retort there are conveyors 11, 12 and 13 installed having movable pulleys 14, 15 and 16, and drive pulleys 17, 18 and 19. On the conveyer band 20 there are lobes of stainless heat-resistant steel fastened, which height reaches the retort body walls with a gap not more than 0,5 mm. Shafts of drive pulleys from the outer side of the retort and the casing are connected to reducers 21, 22, 23, which are set in motion by synchronous or induction motors with changing shaft speed. Inside the retorts across the retort cylinder from the elevated side of the retort there are dephlegmatic lattices 24, 25 and 26 for primary purification installed, consisting of 4 metal membranes having a lot of through apertures more than 6 mm in diameter.

The feeding opening 27 is made on the first stage retort 1 from the conveyer 11 drive side, over the opening a feeding device is assembled for feeding of crushed waste, and consists of the hopper 28 and the batcher 29. In the low part of the retort 1 there is a cofferdam 30 for unloading of the solid residue to the next stage of processing into the retort 2, and in the low part of the retort 2 there is a cofferdam 31 for unloading of the solid residue to the next stage of processing into the retort 3. In the low part of the retort 3 there is a locking discharging device 32 for unloading of pyrographite, and a conveyer 33 which supplies pyrographite to a sorting and packing shop is installed.

The burners for fuel oil and gas burners 34 are installed on the outer casing of each retort. Gas burners have block units for slide automatic power control according to the set temperature in the retort. Interstices between the retorts and outer casings are connected between them sequentially by pipes 35, 36, and a smoke tube 37 is diverted from the first stage casing 5.

Between the retorts there are two dephlegmators 38 and 39 installed which are attached to the cavities of the retorts with pipes and connected between them, so:
the first dephlegmator 38 is connected to the second dephlegmator 39 with the pipe 40, and to the pyrolysis first stage retort 1 with the pipe 41 for gas-vapor mixture, and from it the pipe 42 is directed for supply of gas-vapor mixture to the cooler 43, and from the lowest place the pipe 44 is diverted to the second stage retort cavity 2 for tar composition;
the second dephlegmator 39 is connected to the retorts of the second 2 and the third 3 stage with the pipes 45, 46 for gas-vapor mixture, and from the lowest place the pipe 47 is diverted to the third stage retort cavity 3 for tar composition.

The dephlegmators 38 and 39 are cylinders closed from the ends, assembled at the angle of 20° to horizon, in which twelve steel membranes 48 with a lot of apertures are installed.

The conduit 49 is diverted from the cooler 43 to gas burners.

Here is the particular embodiment of the dephlegmatic phased method of organic waste utilization and operation of the pyrolysis apparatus:
milled organic refuse are being loaded into the hopper 28, from which they get to the conveyer 11 of the first stage retort 1 and being exposed to the pyrolysis at the temperature 150-180° C., depending on organic waste composition;
non-decomposed solid residue from the first stage reactor retort 1 is being conveyed to the second stage retort 2, and gas-vapor mixture through the dephlegmatic lattices for primary purification 24 is being conveyed to the first dephlegmator 38, the mixture is purified from tar composition, and tar composition gathered on lattices for primary purification is being exposed to re-pyrolysis in the first stage retort 1, and from the dephlegmator 38 tar composition is being conveyed to the second stage retort 2, gas-vapor mixture from the dephlegmator 38 is being conveyed for cooling to the fraction distributor 43, and combustion gases from the cavity 8 of the casing 5 are being directed outside together with combustion gases generated by the heaters under the casings of all retorts;
the solid residue obtained from the first stage retort 1 is exposed to the pyrolysis at the temperature 300-400° C. in the retort of the second stage 2, from which non-decomposed solid residue is being conveyed to the third stage retort 3 for pyrolysis at the temperature 500-900° C.;
gas-vapor mixture from the retorts of the second 2 and the third 3 stages is being conveyed to the second dephlegmator 39, purified from tar composition, tar composition is being conveyed to a retort of the third stage 3, and the purified gas-vapor mixture from the second dephlegmator 39 is being introduced to the first dephlegmator, purified from tar composition, and tar composition is conveyed to the second stage retort. In the second and the third stages retorts gas-vapor mixture is being purified additionally on the dephlegmatic lattices for primary purification, tar composition is being conveyed to the third stage retort 3, and gas-vapor mixture being conveyed to the dephlegmator 39, tar composition from which returns to the third stage retort 3;
from the fraction distributor 43 gas is being introduced to the reservoir for collection and is being used for the burners for heating of the retorts 34, liquid is being transferred to reservoirs, and solid residue from the last retort is being transported for cooling and to the packing shop by the conveyer 33 through the discharging device 32;
combustion gases from the cavity 10 by the pipe 36 are being conveyed to the cavity 9, wherein they return heat, and then are directed to heat recovery and purification outside the aggregate.

In the description of the invention and operation of the apparatus it is shown the wide range of the pyrolysis temperatures in each retort, as the apparatus is intended for pyrolysis of different and in each case various organic materials with different optimal pyrolysis temperatures, but:
the first stage of pyrolysis is executed at the temperatures 150-180° C. to provide processing of the lightest fractions without overheating and unjustified loss for heating, for such heating in most cases heat from effluent combustion gases from cavities of heating of the next stages retorts is enough;
the second stage is executed at the temperature not higher than 400° C. to provide for a possibility for making of retorts both of the first and the second stages and their saturation from ordinary boiler steel, not from heat-resistant steel being used for making of the third stage retort and its saturation only;
phased heating of solid residue in different retorts with the re-pyrolysis at higher temperature, and the re-pyrolysis of tar composition at higher temperature provide for obtaining of qualitative fuel oil and gas with the necessary molecular mass.

1. A dephlegmatic phased method of organic waste utilization comprising multistage pyrolysis of waste in a reactor without air access at a temperature not more than 1000° with obtaining of solid carbon residue and multi-component gas-vapor mixture, cooling of gas-vapor mixture in multiloop circulatory cooling system, extraction of a heavy fraction for a re-pyrolysis and obtaining of a liquid fraction having specified molecular mass and gas on a finite circuit, wherein said organic waste is exposed stepwise to the pyrolysis with reloading of non-decomposed solid residue from a first stage retort having temperature 150-180° C., sequentially to retorts for the pyrolysis at higher temperatures, and said gas-vapor mixture from said retorts is directed to not less than one dephlegmator, wherein in said dephlegmator said gas-vapor mixture is purified from a tar composition, said tar composition being returned to the retort for the re-pyrolysis, said gas-vapor mixture from the dephlegmator being conveyed to a fraction distributor for cooling, and wherein combustion gases from a casing of the last retort being conveyed to casings of the preceding retorts, and then jointly with combustion gases generated by heaters under casings of all retorts are delivered outside.

2. A method according to claim 1, wherein said non-decomposed solid residue from the first stage retort having temperature 150-180° C., is being conveyed to a second stage retort for the pyrolysis at temperature 390-400° C., from which said non-decomposed solid residue is being conveyed to a third stage retort for the pyrolysis at temperature 500-900° C., and said gas-vapor mixture purified from the tar composition from the first retort is being conveyed to a first dephlegmator, and said tar composition being conveyed to the second stage retort, and wherein said gas-vapor mixture purified from said tar composition from said second and third stage retorts being directed to a second dephlegmator, and the tar composition being conveyed to the third stage retort, and wherein purified gas-vapor mixture from said second dephlegmator is conveyed to said first dephlegmator, and the tar composition is conveyed to said second stage retort.

3. A method according to claim 2, said the gas-vapor mixture of the second stage retort is being purified on dephlegmatic lattices of said retort, and the tar composition obtained being conveyed to the third stage retort, said gas-vapor mixture of the first stage retort being conveyed to a dephlegmatic lattices of said first stage retort, and the tar composition obtained is being returned to the second stage retort.

4. A method according to claims 1, wherein from said fractions distributor gas is conveyed to a reservoir for collection used for retort heating burners, said liquid fraction is transferred to reservoirs and said solid residue from the last retort is conveyed for cooling to a packing shop through a discharging device.

5. A method according to claims 1, wherein said combustion gases are being directed for heat recovery and for purification outside an aggregate.

6. A dephlegmatic pyrolysis apparatus for organic waste utilization, comprising a pyrolysis reactor with external heating, a feeding device, a system of burners, gas main line having feeding facilities of the system of burners, a system for outlet of combustion gases from the heating system and pyrolysis gases from the pyrolysis reactor, a discharging device, said reactor has a device for moving waste from a filling zone to a discharging zone during the time of a process cycle, wherein all parts of said apparatus are made of heat-proof and resistant to corrosive environment material, wherein said apparatus contains not less than two cylindrical retorts which are assembled on a frame inside an external housings forming a furnace cavity and upon 20°-40° tilt of a cylinder generatrix relative to a horizon, and wherein said apparatus contains not less than one dephlegmator which is a cylinder closed from both ends and assembled at an angle of 20°-40° relative to a horizon, said dephlegmator internally equipped with numerous pierced metal membranes across said cylinder, wherein inside of each retort there are conveyers with movable and drive pulleys, said conveyers are provided with lobes attached to a conveyer band raking up raw material from an inner cylindrical surface of a respective retort casing, wherein each retort has an opening located in a lowest part provided for discharge of solid residue to the next stage of processing into the following retort, and wherein said dephlegmator is attached to said openings with pipes.

7. Apparatus according to claim 6, wherein inside of each of said retorts behind said movable pulley across the retort cylinder from the elevated side of the retort there are installed dephlegmatic lattices for primary purification, said dephlegmatic lattices are consisting of 4-5 metal membranes provided with a numerous through apertures.

8. Apparatus according to claims 6, further comprising three retorts installed on a frame and two dephlegmators which are attached by pipes to the retort's cavities and connected between them, wherein a first dephlegmator is connected to a second and a first stage retort by means of a pipe for gas-vapor mixture, said pipe is conveying said gas-vapor mixture to a cooler, and wherein from the lowest place said pipe is diverted to the second stage retort's cavity, and wherein a second dephlegmator is connected to the second and a third stage retorts by means of a pipe for gas-vapor mixture, said pipe from the lowest place of said dephlegmator is diverted to the third stage retort cavity.

9. Apparatus according to claim 6, wherein under an outer casing of each retort there are installed gas burners, block units provided with a slide automatic power control to set temperature in each of said retort and burners for fuel oil with three-stage power control, wherein cavities between said retorts and outer casings are connected with pipes and a smoke tube diverted from the first stage retort casing.

10. Apparatus according to claim 6, wherein each retort is provided with ribbing formed on the outer side of said retorts along the entire area of the retort's cylinder generatrix.

11. Apparatus according to claims 6, wherein said first and second stages retorts and their saturation are made of ordinary boiler steel.
